(19) United States
(12) Patent Application Publication
(10) Pub. No.: US 2025/0195437 A1
(43) Pub. Date: Jun. 19, 2025

(54) CONTINUOUS SPIN FREEZE-DRYING OF NUCLEIC ACID CONTAINING COMPOSITIONS

(71) Applicant: eTheRNA immunotherapies NV, Niel (BE)

(72) Inventors: Iris BOMBEKE, Niel (BE); Katarina BUKARA, Niel (BE); Phillip CHALLIS, Niel (BE); George R. DAKWAR, Niel (BE); Senne DILLEN, Niel (BE); Diletta ESPOSITO, Neil (BE); Eva KICKOVÁ, Niel (BE); Sarah VANDENBERGHE, Niel (BE); Saverio VENDEMIA, Niel (BE)

(21) Appl. No.: 18/846,852

(22) PCT Filed: Mar. 15, 2023
(86) PCT No.: PCT/EP2023/056570

§ 371 Date: Sep. 13, 2024

(30) Foreign Application Priority Data
Mar. 15, 2022 (EP) 22162013.1

(51) Int. Cl. A61K 9/19; A61K 9/51; A61K 31/7105; A61K 47/26

(52) U.S. Cl. CPC A61K 9/19; A61K 9/5123; A61K 31/7105; A61K 47/26

(57) ABSTRACT

The present invention relates to the field of freeze-drying compositions, in particular nucleic acid containing compositions. More specifically, the present invention relates to freeze-dry nucleic acid containing compositions using a method of continuous spin freezing and continuous spin drying (sublimation and desorption) the composition. The invention further relates to a nucleic acid containing composition by making use of the method according to the invention.

### FIELD OF THE INVENTION

The present invention relates to the field of freeze-drying compositions, in particular nucleic acid containing compositions. More specifically, the present invention relates to freeze-drying nucleic acid containing compositions using a method of continuous spin freezing and continuous drying (sublimation and desorption) of the composition. The invention further relates to a nucleic acid containing composition obtainable by the method according to the invention.

### BACKGROUND TO THE INVENTION

Most biopharmaceutical products have a limited stability in an aqueous solution which might result in less potency or even in toxicity of the molecules. In particular, nucleic acids including DNA and RNA are very instable in aqueous solutions at ambient temperatures for long periods because of degradation by contaminating nucleases and because of inherent chemical instability. This instability and loss of integrity is a bottleneck for the application of nucleic acid-based products in the pharmaceutical and biotechnology industry.

Freeze drying (also known as lyophilisation) is applied to maintain a product's shelf life, making it more convenient to be stored, distributed, and transported. A freeze-drying process typically comprises three consecutive steps: i) a freezing step wherein water crystallizes to ice, ii) a primary drying step wherein ice crystals are removed under vacuum by sublimation, iii) a secondary drying step wherein most of the unfrozen water is removed by diffusion and desorption. Although the traditional ‘batch’ freeze-drying can process a large number of vials in one step (e.g. freezing), this method is time- and energy-consuming. The major drawbacks are both the uncontrolled freezing and the uneven heat transfer culminating different process conditions for each individual vial in the batch, leading to uncontrolled vial-to-vial and batch-to-batch variability.

To overcome the shortcomings related to traditional batch freeze-drying, a continuous freeze-drying technology has been developed in which all freeze-drying steps i) to iii) are integrated in a single production line where the selected vials filled with the correspondent liquid formulations can be placed in a continuous mode. One important difference is that the vials, filled with a liquid product, are rapidly rotated along their longitudinal axis and simultaneously cooled (i.e. spin-freezing). Consequently, the resulting frozen product will be spread with a uniform thickness over the entire vial surface (i.e. large surface area and thin product layer). An appropriate load-lock system may be used to rapidly transfer the frozen vials between the continuous freezing and the continuous drying unit, without affecting the specific conditions of pressure and temperature. In the drying chamber, an endless belt system allows the transport of the vials in front of individually controlled radiators which provide the heat transfer to the vials needed for sublimation and desorption, hence allowing individual vial temperature-regulation, which is an advantage compared to existing freeze-drying technologies. A condenser system is used allowing it to continuously remove the condensed water.

Overall, this continuous integrated approach strongly reduces the variability of critical quality attributes and consistently guarantees the predefined quality of the spin freeze-dried product. Spin freezing and drying cycles decrease the exposure of the product to potentially destabilizing temperatures. This is valid for formulated nucleic acids and for nucleic acids in their naked form (i.e. unprotected nucleic acid such as produced in the laboratory). Hence, we found that spin-freeze drying nucleic acid containing compositions enhances stability of the product due to a shorter processing time and process uniformity.

Typically for freeze-drying of nucleic acid containing compositions, classical freeze-drying using rather low concentrations of cryoprotectants are used (WO2011069528). Moreover, whereas continuous spin-freeze-drying of unit doses has already been disclosed in Leys et al., 2020, herein no nucleic-acid containing compositions have been freeze-dried, nor higher concentrations of cryoprotectants were used (Leys et al., 2020—European Journal of Pharmaceutics and Biopharmaceutics—A primary drying model-based comparison of conventional batch freeze-drying to continuous spin-freeze-drying for unit doses). Since the cryoprotectant concentration may significantly impact the quality and characteristics of the freeze-dried nucleic acid containing cakes, the inventors sought to provide a freeze-drying method which allowed the highest flexibility in varying the cryoprotectant concentration. Thereto, it was found that the spin-freeze drying method of the present invention allowed a lot more flexibility on the cryoprotectant concentration compared to the classical freeze-drying methods. In particular, concentrations of cryoprotectant of 20% w/v negatively impacted cake quality in the classical freeze-drying methods, whereas similar concentration of cryoprotectant maintained good cake quality in the spin-freeze drying method of the invention.

It was thus unexpectedly found that spin freezing and drying cycles are beneficial for nucleic acids and nucleic acids containing compositions, in particular when using varying concentrations of cryoprotectants. Specifically, spin freezing generates a homogenous thin layer on the vial inner wall and prevents/hampers the structural re-organization on the nucleic acid level or nucleic acid containing compositions to occur around the glass transition temperature (Tg′) and maintains the original configuration of naked and formulated nucleic acids similar to that of liquid state. Consequently, the product is not destabilized and preserves its critical quality attributes, as well as its biological activity.

### SUMMARY OF THE INVENTION

In a first aspect, the present invention provides a method for freeze-drying a nucleic acid containing composition, the method comprising the steps of:
a) loading a container having an inner and an outer wall with a nucleic acid containing liquid formulation; in particular, a nucleic acid containing liquid formulation comprising above 15% (w/v) of cryoprotectant.
b) spinning said container of step a) along an axis, thereby allowing said formulation to form a layer at the circumferential inner wall of said container;
c) while continuing spinning said container of step b), cooling said container to freeze-dry said formulation at the circumferential inner wall; thereby obtaining a spin-frozen container;
d) drying said spin-frozen container of step c) by subjecting said container to a heating source which provides a homogenous heat transfer to the circumferential inner wall of the container, to form a dried nucleic acid containing composition.

In a specific embodiment of the present invention, said nucleic acid containing composition further comprises one or more lipids, liposomes, lipid nanoparticles, polymers, polymer-based nanoparticles, buffer/media, water for injection, Tris-buffered saline (TBS), and/or phosphate buffered saline (PBS).

In another specific embodiment, said nucleic acid containing liquid formulation further comprises a cryoprotectant. In particular, the cryoprotectant of the present invention may be selected from the list comprising sodium citrate, sodium chloride, sorbitol, polysorbate, trehalose, mannose, mannitol, maltose, sucrose, glucose, fructose, lactose, histidine, arginine, lysine, dextran, maltodextrin, cyclodextrins, polyvinylpyrrolidone (PVP), glycine, glycerol, polyethylene glycol (PEG), propylene glycol, and/or mixtures thereof; in particular sucrose.

In yet a further embodiment of the present invention, said nucleic acid containing liquid formulation comprises a concentration of said cryoprotectant ranging from 0.01% to 25% (w/v) or 0.01% to 25% w/v; more in particular from 16% to 25% (w/v).

In a further embodiment of the method of the present invention, in steps b) and c) said container is spinned along its longitudinal axis at about and between 1000 and 5000 rpm.

In a further embodiment of the method of the present invention, step c) comprises the use of an inert cryogenic gas flux;

In yet a further embodiment, the method of the present invention is performed in a 2-chamber system comprising a freezing chamber and a drying chamber.

In another particular embodiment, said spin-frozen container of step c) is rapidly transferred from said freezing chamber to said drying chamber through an intermediate load-lock compartment, such that the system allows for a continuous freeze-drying method. In particular, said load-lock compartment may maintain a pressure of between about 5 Pa to 35 Pa and a temperature of between about −5° C. to −30° C. in each chamber;

In a specific embodiment of the present invention, said drying chamber is an endless belt system that transports said spin-frozen container in front of at least one individually controlled non-contact heating source; in particular said individually controlled heating source may be selected from an infrared radiator and/or an electrical heating pad.

In a further embodiment, said spin-frozen container slowly rotates along its longitudinal axis at about 5-12 rpm in front of the at least one individually controlled heating source for a time between about 1 and 120 minutes;

In yet a further embodiment of the method of the present invention, step d) comprises a primary drying step where ice crystals are removed via sublimation under vacuum; and a secondary drying step where most of the remaining unfrozen liquid is removed by diffusion and desorption.

In particular, said sublimated ice crystals and desorbed liquid are continuously removed using cryogenic ice condensers.

In a further aspect, the present invention provides for a freeze-dried nucleic acid containing composition obtainable by the methods as disclosed herein.

In particular, the present invention provides for a container comprising a nucleic acid-containing composition in the form of a powder, wherein said powder is in the form of a thin layer at the circumferential inner surface of said container.

### BRIEF DESCRIPTION OF THE DRAWINGS

With specific reference now to the figures, it is stressed that the particulars shown are by way of example and for purposes of illustrative discussion of the different embodiments of the present invention only. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings making apparent to those skilled in the art how the several forms of the invention may be embodied in practice.

FIG. 1: Illustration of the spin-freezing step

Containers filled with a nucleic acid containing liquid formulation are rapidly rotated along their longitudinal axis to form a thin layer of product which is spread over the entire inner vial wall (i.e., spin-freezing). When a homogeneous product layer is obtained, the flow of a cold, inert and sterile gas leads to the cooling and freezing of the solution and solidifies the nucleic acid containing formulation over the entire inner container wall resulting in a thin product layer of a uniform thickness.

FIG. 2: Continuous freezing system connected to a continuous drying system

Containers that were spin-frozen in a continuous fashion are transported to a continuous drying chamber via a load-lock system. Load-lock systems connect the freezing and drying chambers while maintaining specific conditions of pressure and temperature.

FIG. 3: Illustration of infrared-assisted continuous primary drying of spin-frozen vials rotating along their longitudinal axis in front of individual infrared heaters

In the drying chamber, an endless belt system allows the transport of the spin-frozen containers in front of individually controlled radiators which provide a uniform and adequate heat transfer to the entire container surface to achieve an efficient and homogeneous drying behaviour. Each container is rotating very slowly in front of a single radiator, hence, allowing for individual temperature-regulation which enables an optimal drying trajectory for each spin-frozen container.

FIG. 4: Product temperature profile during freezing process

Freezing is the first step in the freeze-drying process, which solidifies the materials. The figure depicts a change in temperature at a cooling rate of 15° C./min (° C., y-axis) in function of time (minutes, x-axis) of the freezing step. The thin line represents the temperature setpoint of the freeze cycle whereas the thick line the actual product temperature. The arrows indicate the moment (t=7 min) that the temperature of the product completely stabilizes to the setpoint temperature. The rate of freezing is important for the formation and size of ice crystals—fast rate of freezing forming smaller ice crystals and vice versa.

FIG. 5: Product temperature profile during drying process

The drying phase of the freeze-dry process comprises two steps, i.e. a primary and a secondary step. The figure depicts a change in temperature (° C., y-axis) in function of time (minutes, x-axis) for the drying phase. The arrow (t=0-40 min) indicates the temperature which separates the primary drying phase (sublimation) from the secondary drying phase (desorption).

FIG. 6: Visual aspect of the product before and after reconstitution of the cake in water for injection

The obtained dried product forms a thin layer which is homogenously distributed around the inner vial wall. Upon addition of 500 μL water for injection to reconstitute the powder, a white to off-white translucent liquid was obtained which is comparable to the visual aspect of the liquid before freeze-drying. The 10× diluted product resulted in a clear solution. Both the reconstituted and 10× diluted liquid were free of visible particles.

The particle size was slightly increased upon reconstitution of the dried product, however, the reconstituted sample demonstrated high particle size homogeneity (i.e., narrow size distribution) which is reflected in the low polydispersity index value.

FIG. 7: Visual appearance of the FD cake by conventional (left panels) vs spin freeze drying (right panels) using 15% w/v sucrose (A) or 20% w/v sucrose (B). For conventional freeze-drying (left panels) 15% w/v sucrose (A) results in an FD cake which visually looks perfect, homogeneous and has no cracks. On the other hand, using 20% w/v sucrose (B) results in an FD cake of bad quality, which visually shows shrinkage, is fragile, shows some cracking, and some crystallization on the bottom of the vial. For spin-freeze drying (right panels) both 15% w/v (A) and 20% w/v (B) results in a perfect FD cake which is homogeneous, without cracks.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

The present invention thus in particular relates to a freeze-drying process for nucleic acid containing compositions making use of a spin-freeze drying step. The inventors of the present invention have found that spin freeze drying nucleic acid containing compositions have a better stability for distribution and storage compared to freeze-dried compositions using standard freeze-drying methods.

A further advantage of the present invention is that the freeze-drying method described in the present invention is 20-40 folds faster compared to the conventional freeze-drying. This results in a significant reduction of the formation of ice crystals.

Accordingly, and as already detailed herein above, in a first aspect, the present invention provides a method of freeze-drying a nucleic acid containing composition, the method comprising the steps of:
a) loading a container having an inner and an outer wall with a nucleic acid containing liquid formulation; in particular a nucleic acid containing liquid formulation comprising above 15% (w/v) of cryoprotectant;
b) spinning said container of step a) along an axis, thereby allowing said formulation to form a layer at the circumferential inner wall of said container;
c) while continuing spinning said container of step b), cooling said container to freeze-dry said formulation at the circumferential inner wall; thereby obtaining a spin-frozen container;
d) drying said spin-frozen container of step c) by subjecting said container to a heating source which provides a homogenous heat transfer to the circumferential inner wall of the container, to form a dried nucleic acid containing composition.

As used herein and unless otherwise specified, the term “nucleic acids” is to be understood as biopolymers, or large biomolecules, composed of nucleotides (which are the monomers made of three components: a 5-carbon sugar, a phosphate group and a nitrogenous base). The term “nucleic acid” is the overall name for information-carrying molecules such as RNA (ribonucleic acid; sugar compound is ribose) and DNA (deoxyribonucleic acid; sugar compound is deoxyribose). In the present invention, the sequences in question can be of natural (human, animal, vegetable, bacterial, viral) or artificial origin, and in particular genomic DNA, cDNA, messenger RNA (mRNA), transfer RNA (tRNA), ribosomal RNA (rRNA), microRNA (miRNA), small interfering (siRNA), small nucleolar RNA (snoRNA), piwi-interacting RNA (piRNA), tRNA-derived small RNA (tsRNA), small rDNA-derived RNA (srRNA), hybrid sequences or synthetic or semi-synthetic sequences. The term naked nucleic acids refer to unprotected nucleic acids such as DNA/RNA produced in the laboratory. In addition, the nucleic acid can vary in size, ranging from oligonucleotide to chromosome and may be single- or double-stranded.

In the context of the present invention, the term “RNA” relates to a molecule which comprises ribonucleotide residues and preferably being entirely or substantially composed of ribonucleotide residues. “Ribonucleotide” relates to a nucleotide with a hydroxyl group at the 2′-position of a P-D-ribofuranosyl group. The term includes double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations can include addition of non-nucleotide material, such as to the end(s) of a RNA or internally, for example at one or more nucleotides of the RNA. Nucleotides in RNA molecules can also comprise non-standard nucleotides, such as non-naturally occurring nucleotides or chemically synthesized nucleotides or deoxynucleotides. These altered RNAs can be referred to as analogs. Nucleic acids may be comprised in a vector. The term “vector” as used herein includes any vectors known to the skilled person including plasmid vectors, cosmid vectors, phage vectors such as lambda phage, viral vectors such as adenoviral or baculoviral vectors, or artificial chromosome vectors such as bacterial artificial chromosomes (BAC), yeast artificial or analogs of naturally-occurring RNA.

According to the present invention, the term “RNA” includes and preferably relates to “mRNA” which means “messenger RNA” and relates to a “transcript” which may be produced using DNA as template and encodes a peptide or protein. mRNA typically comprises a 5′ untranslated region (5′-UTR), a protein or peptide coding region and a 3′ untranslated region (3′-UTR). mRNA has a limited halftime in cells and in vitro. Preferably, mRNA is produced by in vitro transcription using a DNA template. In one embodiment of the invention, the RNA is obtained by in vitro transcription or chemical synthesis. The in vitro transcription methodology is known to the skilled person. For example, there is a variety of in vitro transcription kits commercially available. In a specific embodiment of the present invention, said mRNA molecules are mRNA molecules encoding immune modulating proteins.

In the context of the present invention, the term “mRNA molecules encoding immune modulating proteins” is meant to be mRNA molecules encoding proteins that modify the functionality of antigen presenting cells; more in particular dendritic cells. Such molecules may be selected from the list comprising CD40L, CD70, caTLR4, IL-12p70, EL-selectin, CCR7, and/or 4-1 BBL, ICOSL, OX40L, IL-21; more in particular one or more of CD40L, CD70 and caTLR4. A preferred combination of immunostimulatory factors used in the methods of the invention is CD40L and caTLR4 (i.e. “DiMix”). In another preferred embodiment, the combination of CD40L, CD70 and caTLR4 immunostimulatory molecules is used, which is herein also named “TriMix”.

In another specific embodiment, said mRNA molecules are mRNA molecules encoding antigen- and/or disease-specific proteins.

According to the present invention, the term “antigen” comprises any molecule, preferably a peptide or protein, which comprises at least one epitope that will elicit an immune response and/or against which an immune response is directed; accordingly, the term antigen is also meant to encompass minimal epitopes from antigens. A “minimal epitope” as defined herein is meant to be the smallest structure which is capable of eliciting an immune response. Preferably, an antigen in the context of the present invention is a molecule which, optionally after processing, induces an immune response, which is preferably specific for the antigen or cells expressing the antigen. In particular, an “antigen” relates to a molecule which, optionally after processing, is presented by MHC molecules and reacts specifically with T lymphocytes (T cells).

In a specific embodiment, the antigen is a target-specific antigen which can be a tumor antigen, or a bacterial, viral or fungal antigen. Said target-specific antigen can be derived from either one of: total mRNA isolated from (a) target cell(s), one or more specific target mRNA molecules, protein lysates of (a) target cell(s), specific proteins from (a) target cell(s), or a synthetic target-specific peptide or protein and synthetic mRNA or DNA encoding a target-specific antigen or its derived peptides.

To avoid any misunderstanding, the LNPs of the present invention may comprise a single mRNA molecules, or they may comprise multiple mRNA molecules, such as a combination of one or more mRNA molecules encoding immune modulating proteins and/or one or more mRNA molecules encoding antigen- and/or disease-specific proteins.

In a very specific embodiment, said mRNA molecules encoding immunomodulatory molecules may be combined with one or more mRNA molecules encoding antigen- and/or disease-specific proteins. For example, the LNP's of the present invention may comprise mRNA molecules encoding the immunostimulatory molecules CD40L, CD70 and/or caTLR4 (such as Dimix or Trimix); in combination with one or more mRNA molecules encoding antigen- and/or disease-specific proteins. Thus, in a very specific embodiment, the LNP's of the present invention comprise an mRNA molecule encoding CD40L, CD70 and/or caTLR4; in combination with one or more mRNA molecules encoding antigen- and/or disease-specific proteins.

In the context of the present invention, the term “composition” refers to the identity of all different, individual substances, compounds or elements (e.g. agents, modulators, regulators, etc.) that constitute the composition. It can be a solution, a suspension, liquid, powder or a paste, aqueous or non-aqueous formulations or any combination thereof. The compositions are preferably pharmaceutical compositions, comprising one or more pharmaceutically excipients, carriers, diluents.

In a specific embodiment of the present invention, said nucleic acid containing composition further comprises one or more lipids, liposomes, lipid nanoparticles, polymers, polymer-based nanoparticles, buffer/media water for injection, Tris-buffered saline (TBS), and Phosphate buffered saline (PBS).

In the context of the present invention, the term ‘nucleic acid containing composition’ refers to a composition comprising at least one nucleic acid polymer that is delivered in one or more vehicles comprising lipids, liposomes, lipid nanoparticles, polymers, polymer-based nanoparticles, buffer/media water for injection, Tris-buffered saline (TBS), and/or Phosphate buffered saline (PBS).

After freeze-drying, the resulting dried nucleic acids may also again be reconstituted using any suitable medium/buffer, such as but not limited to water for injection, Tris buffered saline (TBS) and/or Phosphate buffered saline (PBS).

As used herein, lipids are referred to as any component from the category fatty acids, glycerolipids, glycerophopholipids, sphingolipids, sterols, prenols, saccharolipids, polyketides. Further, in the context of the present invention, liposomes are spherical vesicle structures having at least one lipid bilayer that forms in the shape of a hollow sphere encompassing an aqueous phase. As such, any cargo of interest such as nucleic acid containing formulations, pharmaceutical drugs, proteins/peptides can be encapsulated within liposomes in either the aqueous compartment (if it is water-soluble/hydrophilic) or within the lipid bilayer (if fat-soluble/lipophilic).

Furthermore, as used herein, lipid nanoparticles are liposome-like structures especially geared towards encapsulating a broad variety of nucleic acids (RNA and DNA) as a drug or vaccine, in a non-aqueous core. More specifically, the lipid nanoparticle is generally spherical in shape and consists of a solid lipid core stabilized by a surfactant. The core lipids can be fatty acids, acylglycerols, waxes, and mixtures of thereof Biological membrane lipids such as phospholipids, sphingomyelins, bile salts (sodium taurocholate), and sterols (cholesterol) can be utilized as stabilizers. While many different types of lipids may be included in such LNP, the LNP's of the present invention are typically composed of a combination of an ionizable lipid, a phospholipid, a sterol, and a PEG lipid.

In the context of the present invention, the term “PEG lipid” or alternatively “PEGylated lipid” is meant to be any suitable lipid modified with a PEG (polyethylene glycol) group.

In the context of the present invention the term “ionizable” (or alternatively cationic) in the context of a compound or lipid means the presence of any uncharged group in said compound or lipid which is capable of dissociating by yielding an ion (usually an H⁺ ion) and thus itself becoming positively charged. Alternatively, any uncharged group in said compound or lipid may yield an electron and thus becoming negatively charged.

In the context of the present invention, the term “phospholipid” is meant to be a lipid molecule consisting of two hydrophobic fatty acid “tails” and a hydrophilic “head” consisting of a phosphate group. The two components are most often joined together by a glycerol molecule, hence, in the phospholipid of the present invention is preferably a glycerol-phospholipid. Furthermore, the phosphate group is often modified with simple organic molecules such as choline (i.e. rendering a phosphocholine) or ethanolamine (i.e. rendering a phosphoethanolamine).

In the context of the present invention, the term “sterol”, also known as steroid alcohol, is a subgroup of steroids that occur naturally in plants, animal and fungi, or can be produced by some bacteria. In the context of the present invention, any suitable sterol may be used, such as selected from the list comprising cholesterol, ergosterol, campesterol, oxysterol, antrosterol, desmosterol, nicasterol, sitosterol and stigmasterol, preferably cholesterol.

As used herein, polymers are referred to as a natural or synthetic substance or material consisting of very large molecules, or macromolecules, composed of many repeating subunits called monomers. The term ‘polymeric nanoparticles’ is to be understood as spherical particles within the size range from 1 to 1000 nm that can be loaded with active compounds entrapped within or surface-adsorbed onto the polymeric core. Further, the term “nanoparticle” is to be understood as both nanocapsules or nanospheres wherein nanocapsules are composed of an oily core in which the drug is usually dissolved, surrounded by a polymeric shell while nanospheres are based on a continuous polymeric network in which the drug can be retained inside or adsorbed onto their surface.

As used herein and unless otherwise specified, the term “container” is to be understood as any receptacle or enclosure for holding a product, that keeps the product protected by being inside of its structure. The container can be made up of glass, plastic, or any other material and is used for storage, package, transport of liquid, powder, tablet or capsule products and may be in the form of for example a vial, bottle, vessel, canister, flask, preferably a cylindrical vial.

As used herein and unless otherwise specified, the term “liquid formulation” is to be understood as any excipient in a liquid or semi-liquid form that comprises at least one component that is either or not dissolved in said liquid or semi-liquid.

In another specific embodiment, said nucleic acid containing liquid formulation further comprises a cryoprotectant. In particular, the cryoprotectant of the present invention may be selected from the list comprising sodium citrate, sodium chloride, sorbitol, polysorbate, trehalose, mannose, mannitol, maltose, sucrose, glucose, fructose, lactose, histidine, arginine, lysine, dextran, maltodextrin, cyclodextrins, polyvinylpyrrolidone (PVP), glycine, glycerol, polyethylene glycol (PEG), propylene glycol, and/or mixtures thereof. In a very specific embodiment the cryoprotectant is sucrose.

As used herein and unless otherwise specified, the term “cryoprotectant” is to be understood as a penetrating or non-penetrating substance used to protect a composition and its associated compounds from freezing damage (i.e. that due to ice formation). Conventional cryoprotectants are commonly glycols (alcohols containing at least two hydroxyl groups) and sugars, such as ethylene glycol, propylene glycol, glycerol and trehalose. In particular, trehalose is a non-reducing sugar consisting of two molecules of glucose that preserves the structural integrity of the cells during freezing and thawing in a non-toxic manner. Previous studies have used this disaccharide for cryopreservation of human cells, such as platelets, red blood cells, sperm, oocytes, pancreatic islets and fetal skin.

In yet a further embodiment of the present invention, said nucleic acid containing liquid formulation comprises a concentration of said cryoprotectant ranging from 0.01% to 25% (w/v) or 0.01% to 25% w/v; in particular from 1% to 20% w/v, more in particular above 5% w/v, more in particular above 10 w/v, more in particular about 15% w/v, more in particular above 15% w/v; more in particular from about 16% to 25% w/v.

In some embodiments, said liquid formulation may comprise sodium citrate with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise sodium chloride with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise sorbitol with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise polysorbate with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise trehalose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise mannose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise mannitol with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise maltose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise sucrose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise glucose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise fructose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise lactose with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise histidine with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise sodium arginine with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise lysine with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise dextran with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise maltodextrin with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise cyclodextrins with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise polyvinylpyrrolidone (PVP) with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise glycine with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise glycerol with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise polyethylene glycol (PEG) with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In some embodiments, said liquid formulation may comprise propylene glycol with a minimum amount of at least about 0.1, 0.2, 0.4, 0.6, 0.8, 1, 2, 3, 4, 5, 6, 7, 8, 9, 10, 11, 12, 13, 14, 15, 16, 17, 18, 19, 20, 21, 22, 23, 24, 25% (w/v).

In a further embodiment of the method of the present invention, during the spinning and cooling step, said container is spinned along its longitudinal axis at about and between 1000 and 5000 rpm.

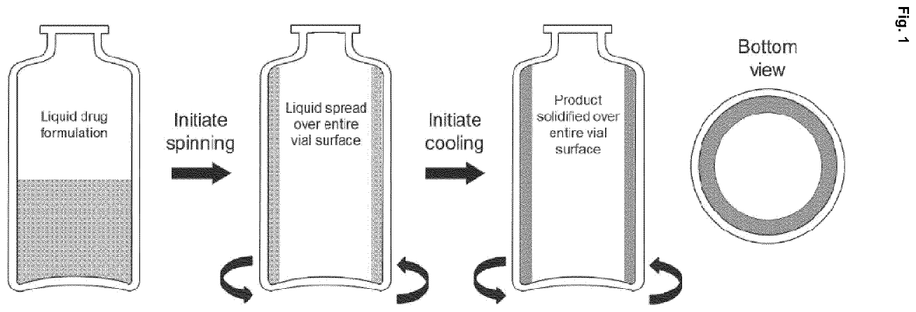

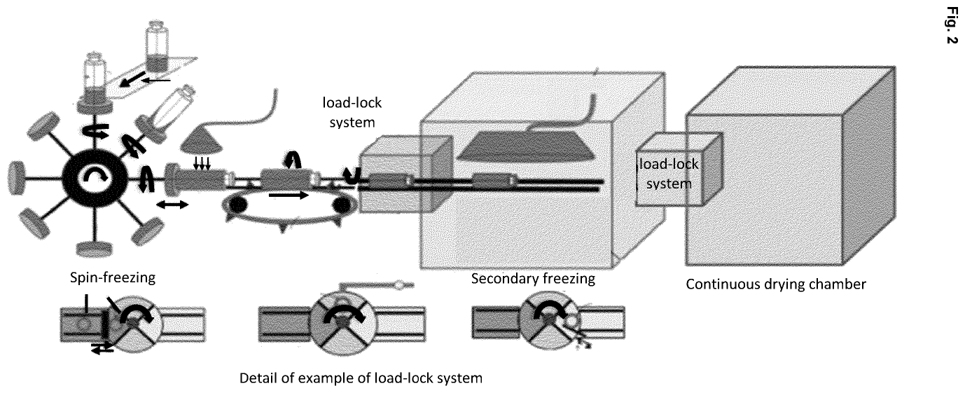

As used herein and unless otherwise specified, the term “spinning” is to be understood as rotating said container around its axis which spreads said formulation as homogenously as possible over the entire inner container surface allowing said formulation to form a layer at the circumferential inner wall of said container (FIG. 1). The spinning (or rotating) of said container may be performed in a continuous way during the freeze-dry process but not necessarily at the same speeds, also referred to as rounds per minutes (rpm).

As used herein and unless otherwise specified, the term “axis” is to be understood as the longitudinal or lateral axis.

In some embodiments, the term “longitudinal axis” is to be understood as the axis going from the top to the bottom of the specified container.

As used herein and unless otherwise specified, the term “lateral axis” is to be understood as the axis that passes horizontally through the center of the specified container.

In some embodiments of the method of the present invention, the spinning of the container during the cooling step is performed at about and between 1000 and 5000 rpm, preferably at about and between 1500 and 4000 rpm, more preferably at about and between 2000 and 3700 rpm.

As used herein and unless otherwise specified, the term “cooling” is to be understood as decreasing the product temperature by a gas in liquid or solid state to below 0° C. typically between about −90° C. to −20° C., more preferably −70° C., to transform the liquid state of the product to a solid state and in particular to form ice crystals at the inner surface of the specified container. The temperature profile during this cooling phase can be dependent on the composition to be cooled and may vary from linear cooling down (for example 15° C./min) to more complex and dynamic temperature profiles. The cooling phase is continued for about 1 to 30 minutes, more preferably 15 minutes.

As used herein and unless otherwise specified, the term ‘ice crystals’ is to be understood as a structure formed at lower temperatures by the condensation of water vapor on a solid body wherein the solid body may be a particle or other substance.

In a further embodiment of the method of the present invention, the cooling step comprises the use of an inert cryogenic gas flux.

As used herein and unless otherwise specified, the inert cryogenic gas flux may be selected from the list comprising: helium, carbon dioxide and nitrogen; preferably carbon dioxide, most preferably nitrogen, which may encircle the at least one container to obtain a thin frozen product layer.

In some embodiments of the method of the present invention, the thin frozen product layer is about 0.1-10 nm in thickness, preferably is about 0.5-5 nm in thickness, most preferably about 1-3 nm in thickness.

In a further embodiment, the rotating or spinning container is continuously cooled for about 1 to 60 minutes, preferably 5 to 30 minutes, more preferably 10 to 20 minutes.

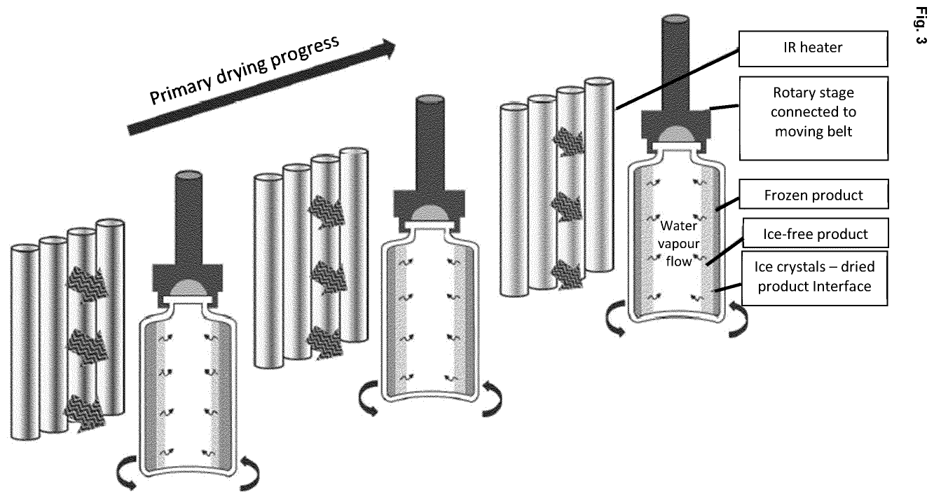

In yet a further embodiment, the method of the present invention is performed in a 2-chamber system comprising a freezing chamber and a drying chamber (FIG. 2).

As used herein and unless otherwise specified, the term “drying” refers to a process in which water or another solvent is removed from a solid, semi-solid or liquid composition. This can be accomplished by subjecting the composition to a heating source, a drying agent, placing the composition under vacuum or a combination thereof.

As used herein and unless otherwise specified, a “2-chamber system” is to be understood as two chambers—a freezing chamber and a drying chamber—that are connected with each other.

In another particular embodiment, said spin-frozen container is rapidly transferred from said freezing chamber to said drying chamber through an intermediate load-lock compartment, such that the system allows for a continuous freeze-drying method (FIG. 2).

In a specific embodiment, said load-lock compartment may maintain a pressure of between about 5 Pa to 35 Pa. In another specific embodiment, said load-lock compartment may have a temperature of between about −5° C. to −30° C. in each chamber. In a further embodiment, said load-lock compartment may maintain a pressure of between about 5 Pa to 35 Pa and a temperature of between about −5° C. to −30° C. in each chamber.

As used herein and unless otherwise specified, the term “load-lock system” is to be understood as a system used to rapidly transfer spin-frozen vials between the continuous freezing and the continuous drying chamber, without disturbing the specific conditions of pressure and temperature in each chamber, guaranteeing the continuity of the process.

In some embodiments of the method of the present invention, the freezing chamber, drying chamber, and load-lock compartments are a closed loop system, wherein the load-lock compartments and drying chamber are preferably under pressure also known as vacuum condition. The container is positioned in a vacuum condition, either subjected to a heating source or not, resulting in a process in which water vapor will be generated wherein the water vapor removed using condensers.

In some embodiments of the method of the present invention, the pressure in the load-lock compartment may be between 0 Pa and 70 Pa, preferably 5 Pa and 40 Pa, most preferably 5 Pa and 35 Pa and can be adapted during the process as such.

In some embodiments of the method of the present invention, the temperature in the load-lock compartment may be between −5 and −30° C., preferably −5 and −25° C., most preferably at about −10° C. and can be adapted during the process as such.

In yet a further embodiment of the method of the present invention, the drying process comprises a primary drying step where ice crystals are removed via sublimation under vacuum; and a secondary drying step where most of the remaining unfrozen liquid is removed by diffusion and desorption. In particular, said sublimated ice crystals and desorbed liquid are continuously removed using cryogenic ice condensers.

In some embodiments, the term “drying” refers to either the term “sublimation” (also referred to as the “primary drying step”) or to the terms “‘diffusion and desorption” (also referred to as the “secondary drying step”), or to both.

As used herein and unless otherwise specified, the term ‘sublimation’ is to be understood as the transition of a substance directly from a solid to a gas state, without passing through a liquid state under vacuum.

In some embodiments of the method of the present invention, the pressure during the sublimation step may be between 0 Pa and 70 Pa, preferably 5 Pa and 40 Pa, most preferably 5 Pa and 35 Pa and can be adapted during the process as such.

As used herein and unless otherwise specified, the term ‘diffusion’ is to be understood as the thermal motion of all (liquid or gas) particles at temperatures above absolute zero under vacuum. Diffusion explains the net flux of molecules from a region of higher concentration to one of lower concentration.

As used herein and unless otherwise specified, the term ‘desorption’ is to be understood as a phenomenon whereby a substance is released from or through a surface into a gas phase under vacuum. In particular, most of the unfrozen water (i.e., water dissolved in the solid amorphous phase) are bound to the interior surface of the container through adsorption and absorption, and gradually removed by desorption.

In some embodiments of the method of the present invention, the pressure during the diffusion and desorption step may be between 0 Pa and 70 Pa, preferably 5 Pa and 40 Pa, most preferably 5 Pa and 35 Pa and can be adapted during the process as such.

As used herein and unless otherwise specified, the term ‘cryogenic ice condenser is to be understood as a system designed to rapidly absorb steam and reduce containment pressure.

In a further embodiment, during the drying process said spin-frozen container slowly rotates along its longitudinal axis at about 5-12 rpm in front of the at least one individually controlled heating source for a time between about 1 and 120 minutes.

In some embodiments of the present invention, continuously spinning of said container may be performed along its longitudinal axis at about and between 150 and 600 rpm/S², preferably at about and between 3 and 25 rpm, more preferably at about and between 5 and 12 rpm.

In some embodiments of the method of the present invention, the primary drying step (also referred to as sublimation), may take up about 1 to 60 minutes, preferably 5 to 30 minutes, more preferably 10 to 20 minutes, most preferably until the residual moisture content is 0 to 30%, preferably, 2 to 25%, more preferably 3 to 20%, most preferably 5 to 15%.

In some embodiments of the method of the present invention, the secondary drying step (also referred to as diffusion and desorption), may take up to about 1 to 60 minutes, preferably 5 to 30 minutes, more preferably 10 to 20 minutes. Alternatively, the secondary drying is continued until the composition has acceptable moisture content for long term storage most preferably a residual moisture content to about 0 to 5%, preferably 0.2 to 4%, most preferably 0.5 to 3%.

In another embodiment of the method of the present invention, the temperature during the secondary drying step may be between 10° C. and 70° C., more preferably 20° C. and 60° C. and most preferably 30° C. and 50° C.

In some embodiments of the method of the present invention, the entire drying step is executed for a period of time of at least 60 minutes, preferably less than or equal to 120 minutes.

In a specific embodiment of the present invention, said drying chamber is an endless belt system that transports said spin-frozen container in front of at least one individually controlled non-contact heating source; in particular said individually controlled heating source may be selected from an infrared radiator and/or an electrical heating pad.

As used herein and unless otherwise specified, the term “endless belt system” is to be understood as a transport system that puts containers in a continuous way in front of a heating source.

As used herein and unless otherwise specified, the term “heating source” is used to indicate a heater which is associated, at least temporarily, with a nearby container, and which is driven to provide heat mainly to said container, preferably without providing heat energy to other containers. The device homogeneously transfers energy either by radiation or conduction for drying (sublimation and desorption) and can be tuned per unit dose.

In some embodiments of the method of the present invention, the heater can be an infrared radiator wherein the infrared heating allows a contactless heat transfer to only opaque objects rather than the air around them.

In another embodiment, the infrared radiator reaches a temperature of about and between 10° and 450° C. and can be adapted during the process as such.

As used herein and unless otherwise specified, the term “infrared”, is also understood as infrared (IR) thermography which allows contactless, real-time and spatial monitoring of the product temperature at the sublimation interface. In a further aspect, the present invention provides for a freeze-dried nucleic acid containing composition obtainable by the methods as disclosed herein.

In particular, the present invention provides for a container comprising a nucleic acid-containing composition in the form of a powder, wherein said powder is in the form of a thin layer at the circumferential inner surface of said container.

### EXAMPLES

### Example 1

### Material and Methods

Lipid nanoparticles containing mRNA were prepared using a mixing device followed by ethanol removal, filtration with 0.22 μm filter and filled in 2 mL type I clear glass vials (fill volume of 0.5 mL per vial).

Drug product formulation containing 500 μL of lipid nanoparticles (comprising an ionisable lipid, a phospholipid, a sterol and a PEG lipid) encapsulating mRNA in Tris-buffered saline (TBS) supplemented with 15% trehalose was subjected to a continuous freeze-drying run on a single vial equipment.

During spin freezing (FIG. 1), the vial was rotated (spinned) around their longitudinal axis at approximately 2500 rotations per minute (rpm), suggested that 2500 rpm resulted in an equally spread product layer with a maximal layer thickness difference of 10% between the bottom and the top of the product layer. The process conditions applied: cooling rate of 15° C./min until −70° C., conservative drying settings, ramp-up during spin-freezing of 150 rpm/s². The drying cycle was performed under conservative settings were the infra-red (IR) heaters (FIG. 3) were switched off at approximately 0.6 Watt, which is the energy value required to sublimate 500 mg of ice. The duration of the freeze-drying cycle was 1.5 hours. Following reconstitution, particle size, polydispersity index and visual aspect were determined.

### Results

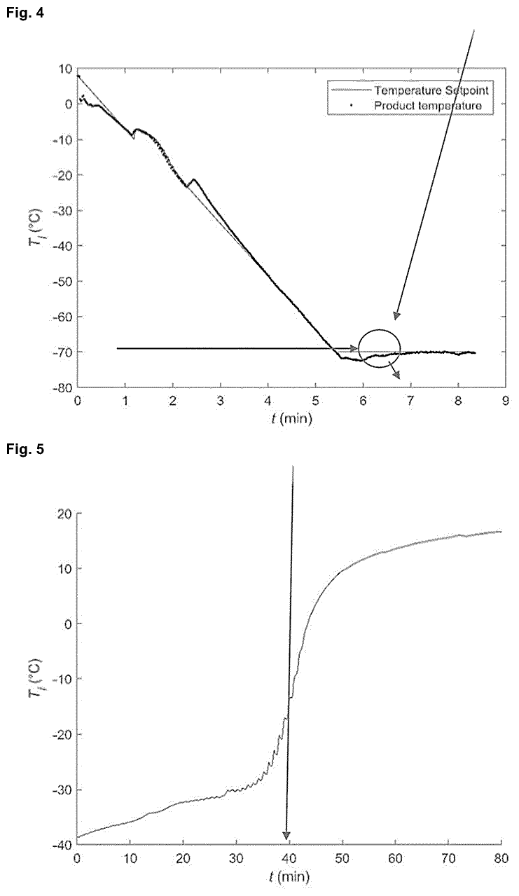

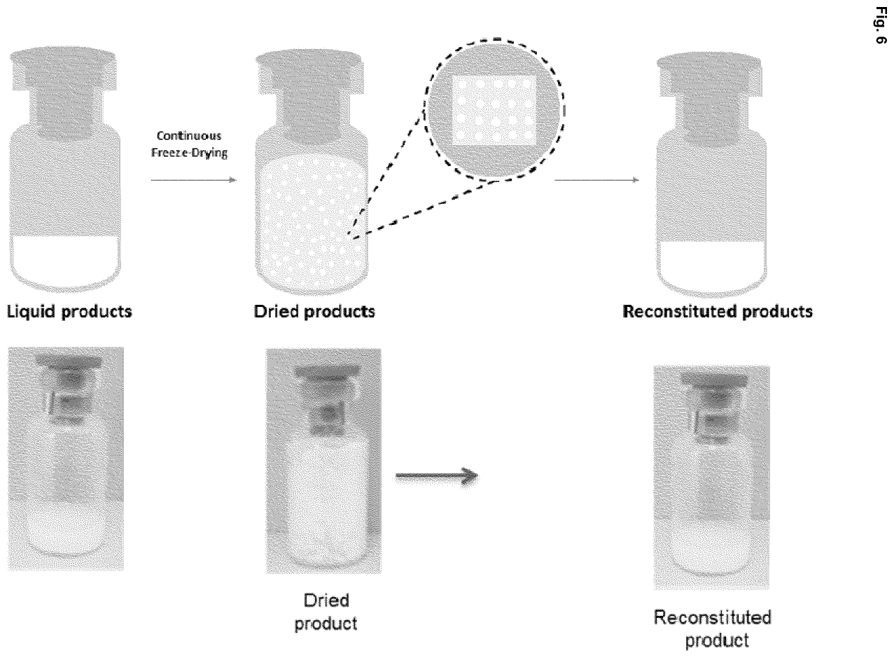

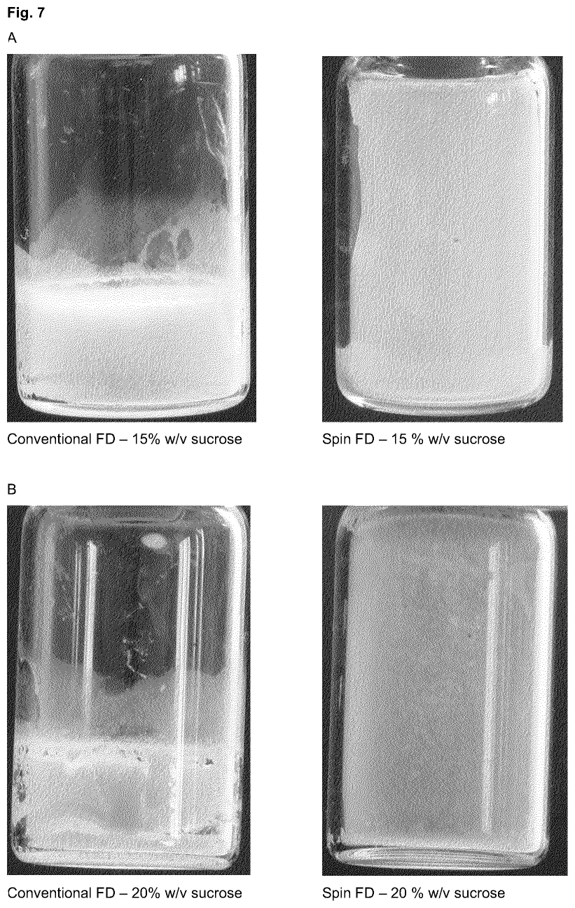

The temperature of the product decreased at a cooling rate of 15° C./min until −70° C. at which the product completely stabilizes to the setpoint temperature (FIG. 4).

FIG. 5 depicts the increase in temperature during the primary and secondary drying phase, respectively left and right from the arrow.

The obtained dried product forms a thin layer which is homogenously distributed around the inner vial wall (FIG. 6). Reconstituting the powder with water forms a white to off-white translucent liquid. The 10× diluted product resulted in a clear solution. Both the reconstituted and 10× diluted liquid were free of visible particles (Table 1). The particle size was slightly increased upon reconstitution of the dried product, however, the reconstituted sample demonstrated high particle size homogeneity (i.e., narrow size distribution) which is reflected in the low polydispersity index value.

TABLE 1
Visual aspects and particle characterization of the dried and reconstituted
product

| | | Particle size | Polydispersity |
|---|---|---|---|
| Process step | Visual aspect | (D_{H} in nm) | index (PDI) |
| Before freeze- | Translucent white to off- | 162 | 0.02 |
| drying | white without visible | | |
| | particles | | |
| Freeze-dried | Homogenous thin layer of | NA | NA |
| product | dried product on the vial | | |
| | wall | | |
| After | Translucent white to off- | 200 | 0.07 |
| reconstitution | white without visible | | |
| | particles | | |

### Example 2

In this example, a comparison of freeze-dried cake in classical freeze-drying versus spin-freeze drying using various concentrations of sucrose was made.

### Lnp Generation:

mRNA in an acidic buffer was mixed with a lipid mixture in ethanol using a T-junction mixer. After mixing, the LNPs dispersion was buffer-exchanged to a neutral buffer using dialysis and then concentrated by centrifugal concentration. Various concentrations of sucrose (5%, 8%, 10%, 15% and 20% w/v) was added to the final LNPs dispersion as cryoprotectant.

### Lyophilization and Analysis:

Conventional lyophilization was performed using a Martin Christ EPSILON 2-6 LSCplus freeze-drier. Spin freeze drying was performed using a RheaVita Multi Vial Unit according to the methods disclosed herein.

After lyophilization, the samples were reconstituted in WFI (Water for injection). The visual appearance of the samples was assessed before and after reconstitution.

### Results

While visual appearance of the lyophilized cake was good for lower levels of cryoprotectant (sucrose), increasing sucrose content above 15% leads to cracking and shrinkage of the cake and signs of melting (see figure. 7 for concentrations 15% and 20% w/v)). Accordingly, spin freeze-drying technology resulted in improved visual aspects and better visual quality of the FD cake when high levels of cryoprotectant are added. All the standard Critical Quality Attributes (CQA) are comparable with minimal differences in size, PDI, pH, osmolality, mRNA content and encapsulation efficiency.

1. A method for freeze-drying a nucleic acid containing composition, the method comprising the steps of:
a) loading a container having an inner and an outerwall with a nucleic acid containing liquid formulation comprising above 15% (w/v) of cryoprotectant;
b) spinning said container of step a) along an axis, thereby allowing said formulation to form a layer at the circumferential inner wall of said container;
c) while continuing spinning said container of step b), cooling said container to freeze-dry said formulation at the circumferential inner wall; thereby obtaining a spin-frozen container;
d) drying said spin-frozen container of step c) by subjecting said container to a heating source which provides a homogenous heat transfer to the circumferential inner wall of the container, to form a dried nucleic acid containing composition.

2. The method according to claim 1, wherein said nucleic acid containing composition further comprises one or more lipids, liposomes, lipid nanoparticles, polymers, polymer-based nanoparticles, buffer/media water for injection, Tris buffered saline (TBS) and/or Phosphate buffered saline (PBS).

3. The method according to anyone of claims 1 or 2, wherein said cryoprotectant is selected from the list comprising sodium citrate, sodium chloride, sorbitol, polysorbate, trehalose, mannose, mannitol, maltose, sucrose, glucose, fructose, lactose, histidine, dextran, maltodextrin, cyclodextrins, polyvinylpyrrolidone (PVP), glycine, glycerol, polyethylene glycol (PEG), propylene glycol, and/or mixtures thereof; in particular sucrose.

4. The method according to claim 3, wherein said nucleic acid containing liquid formulation comprises a concentration of said cryoprotectant ranging from 16% to 25% (w/v).

5. The method according to anyone of claims 1 to 4, wherein in steps b) and c) said container is spinned along its longitudinal axis at about and between 1000 and 5000 rpm.

6. The method according to anyone of claims 1 to 5, wherein step c) comprises the use of an inert cryogenic gas flux;

7. The method according to anyone of claims 1 to 6, wherein said method is performed in a 2-chamber system comprising a freezing chamber and a drying chamber.

8. The method according to claim 7, wherein said spin-frozen container of step c) is rapidly transferred from said freezing chamber to said drying chamber through an intermediate load-lock compartment, such that the system allows for a continuous freeze-drying method.

9. The method according to claim 8, wherein said load-lock compartment maintains a pressure of between about 5 Pa to 35 Pa and a temperature of between about −5° C. to −30° C. in each chamber;

10. The method according to anyone of claims 7 to 9, wherein said drying chamber is an endless belt system that transports said spin-frozen container in front of at least one individually controlled non-contact heating source; in particular said individually controlled heating source is selected from an infrared radiator and/or an electrical heating pad.

11. The method according to claim 10, wherein said spin-frozen container slowly rotates along its longitudinal axis at about 5-12 rpm in front of the at least one individually controlled heating source for a time between about 1 and 120 minutes;

12. The method according to anyone of claims 1-11, wherein step d) comprises a primary drying step where ice crystals are removed via sublimation under vacuum; and a secondary drying step where most of the remaining unfrozen liquid is removed by diffusion and desorption;

13. The method according to claim 12, wherein the sublimated ice crystals and desorbed liquid are continuously removed using cryogenic ice condensers.

14. A freeze-dried nucleic acid containing composition obtainable by the method of anyone of claims 1 to 13.

15. A container comprising a nucleic acid-containing composition in the form of a powder, wherein said powder is in the form of a thin layer at the circumferential inner surface of said container.
